(19) United States
(12) Patent Application Publication
(10) Pub. No.: US 2026/0148832 A1
(43) Pub. Date: May 28, 2026

(54) ELECTRONIC DEVICE FOR MONITORING NUTRITIONAL INTAKE

(71) Applicant: KNU-Industry Cooperation Foundation, Chuncheon-si (KR)

(72) Inventor: Sang Ah LEE, Chuncheon-si (KR)

(73) Assignee: KNU-Industry Cooperation Foundation, Chuncheon-si (KR)

(21) Appl. No.: 18/962,538

(22) Filed: Nov. 27, 2024

(51) Int. Cl. G16H 20/60; G16H 10/00

(52) U.S. Cl. CPC G16H 20/60; G16H 10/00

(57) ABSTRACT

According to an embodiment of the present disclosure, there is provided an electronic device including a memory that stores an AI model for identifying food intake information of a user from food images before and after a meal, and a processor that inputs the food images before and after the meal into the AI model to identify the food intake information of the user, and that monitors health conditions of the user, based on the food intake information.

### TECHNICAL FIELD

The present disclosure relates to an electronic device for monitoring nutritional intake.

### BACKGROUND

A demand for more efficient management of health conditions by using electronic equipment has continuously increased. To follow this trend, various electronic devices for healthcare have been developed to manage exercise, medication, and meals.

Meanwhile, in terms of health management, the meal is a very important item not only for a user who wants to control his or her weight, but also for a patient suffering from a disease.

However, in order to manage the diet in view of individual characteristics such as diseases and health conditions, it is necessary to understand consumed nutritional components consumed through food and to remember his or her own meal menu, which requires a wide variety of efforts. Due to a difficulty in this diet management, there is a very high demand for electronic equipment for supporting the diet management.

However, a method for supporting the diet management through the electronic equipment in the related art is cumbersome, that is, a method in which the users directly input information on their own meal menus, and thus, it is difficult to continuously use the method.

Consequently, a technology is required to identify meal information in a simpler way and to support health management, based on this identified meal information.

(Patent Document 1) Korean Patent No. 10-2544986 (AI Customized Diet and Ingredient Recommendation Service System) is an example in the related art.

### SUMMARY

The present disclosure is devised to easily identify information relating to nutritional components consumed by a user by using only food images before and after meals, and to manage health conditions of the user through this identified information.

Objects of the present disclosure are not limited to objects described above, and other objects and advantages of the present disclosure which are not described may be understood by the following description, and will be more clearly understood by embodiments of the present disclosure. In addition, it will be easily understood that the objects and the advantages of the present disclosure may be realized by means and combinations thereof which are disclosed in the appended claims.

According to an embodiment of the present disclosure, there is provided an electronic device including a memory that stores an AI model for identifying food intake information of a user from food images before and after a meal, and a processor that inputs the food images before and after the meal to the AI model to identify the food intake information of the user, and that monitors health conditions of the user, based on the food intake information.

The processor may determine whether the food images before and after the meal satisfy requirements for identifying the food intake information, based on whether containers and food menus which are included in each of the food images before and after the meal are identical, and may request the user to change the food images before and after the meal, when the requirements are not satisfied.

In addition, in a process of identifying the food intake information of the user from the food images before and after the meal, the processor may identify a mass of food consumed by the user, based on volume information of the reduced food according to a difference between the food image before the meal and the food image after the meal, and may determine the mass of the food consumed by the user by applying density depending on a type of the food to the volume information of the reduced food.

In addition, the processor may identify the mass of the food consumed by the user, based on the volume information of the reduced food according to the difference between the food image before the meal and the food image after the meal, and may determine the mass of the food consumed by the user by reflecting a change in the density according to a cooking method of the food.

In addition, the processor may set a target diet in response to a weight control need of the user, and may generate the target diet in which a numerical value of at least one intake item is increased or decreased from a usual diet of the user, in response to the need.

In addition, the processor may determine information on the usual diet of the user, based on the food intake information of the user which is accumulated over a reference period.

In addition, the processor may calculate a type and a volume of nutritional ingredients consumed by the user, based on the food intake information, and may determine whether the type and the volume of the nutritional ingredients consumed by the user are appropriate.

In addition, the processor may acquire user basic information including at least one of body measurement information, a currently held disease, a gender, and an age of the user, may predict a potential disease in which a probability of a developing disease is equal to or greater than a reference value from the user basic information, and may generate the target diet to prevent the potential disease.

According to another embodiment of the present disclosure, there is provided a system including a user terminal that generates food images before and after a meal through image capturing, and an electronic device that receives the generated food images from the user terminal to analyze the generated food images. The electronic device includes a memory that stores an AI model for identifying food intake information of a user from the food images before and after the meal, and a processor that inputs the food images before and after the meal to the AI model to identify the food intake information of the user, and that monitors health conditions of the user, based on the food intake information.

According to still another embodiment of the present disclosure, there is provided a control method for an electronic device. The control method includes acquiring food images before and after a meal, identifying food intake information of a user by inputting the food images before and after the meal into an AI model, and monitoring health conditions of the user, based on the food intake information.

According to still another embodiment of the present disclosure, there is provided a computer readable medium storing a program that causes a processor of an electronic device to execute the control method.

According to various embodiments of the present disclosure, a type and the amount of food consumed by a user may be identified, based on only food images before and after a meal of the user, and information on nutritional ingredients consumed by the user may be easily confirmed, based on the identified information.

According to various embodiments of the present disclosure, health conditions of the user may be managed through food intake information of the user which is confirmed based on the food images before and after the meal.

### BRIEF DESCRIPTION OF THE DRAWINGS

FIG. 1 is a diagram illustrating a configuration of a system including an electronic device according to an embodiment of the present disclosure.

FIG. 2 is a diagram illustrating a configuration of an electronic device (server) according to an embodiment of the present disclosure.

FIG. 3 is a diagram illustrating a configuration of a diet management unit according to an embodiment of the present disclosure.

FIG. 4 is a diagram illustrating a configuration of a health management unit according to an embodiment of the present disclosure.

### DETAILED DESCRIPTION

Advantages and features of the present disclosure, and a method for achieving the advantages and the features of the present disclosure will become apparent with reference to embodiments described below in detail together with the accompanying drawings. However, the present disclosure is not limited to the embodiments disclosed below, and may be implemented in various different forms. The present embodiments are provided only to ensure that the present disclosure is complete and to fully inform a person skilled in the art, to which the present disclosure belongs, of the scope of the present disclosure, and the present disclosure is defined only by the scope of the appended claims.

Terms used in the present specification are only to describe the embodiments, and are not intended to limit the present disclosure. Herein, a singular expression includes a plural expression unless otherwise specified. Terms “comprises” and/or “comprising” used in the specification do not exclude the presence or the addition of one or more other components except for components specified herein. Like reference numerals refer to like components throughout the specification, and “and/or” includes each combination and one or more combinations of the components specified herein. Although “first”, “second”, and the like are used to describe various components, these components are not limited by these terms. These terms are only used to distinguish one component from another component. Therefore, as a matter of course, a first component specified below may be a second component within the technical scope of the present disclosure.

Unless otherwise defined, all terms (including technical and scientific terms) used in the present specification may be used in a sense commonly understood by a person skilled in the art to which the present disclosure belongs. In addition, terms defined in commonly used dictionaries shall not be ideally or excessively interpreted unless explicitly specifically defined.

The term “unit” or “module” as used in the specification means software or hardware components such as an FPGA or ASIC, and the “unit” or the “module” fulfills certain functions. However, the “unit” or the “module” is not limited to software or hardware. The “unit” or the “module” may be configured to reside on an addressable storage medium, and may be configured to reproduce one or more processors. Accordingly, as an example, the “unit” or the “module” includes components such as software components, object-oriented software components, class components, and task components, processes, functions, attributes, procedures, subroutines, program code segments, drivers, firmware, microcode, circuitry, data, databases, data structures, tables, arrays, and variables. Functions provided in the components and the “units” or the “modules” may be combined into a smaller number of components and “units” or “modules”, or may be further separated into additional components and “units” or “modules.”

Spatially relative terms “below,” “beneath,” “lower,” “above,” “upper,” and the like may be used to easily describe a correlation between one component and another component as illustrated in the drawings. The spatially relative terms should be understood to include mutually different directions of the components when the components are used or operated in addition to the directions illustrated in the drawings. For example, when the component illustrated in the drawings is flipped over, one component described as “below” or “beneath” another component may be placed “above” another component. Accordingly, the exemplary term “below” or “beneath” may include both up and down directions. The components may also be oriented in other directions, and the spatially relative terms may be interpreted according to the orientations.

Referring to the following drawings, an electronic device according to an embodiment of the present disclosure will be described in detail.

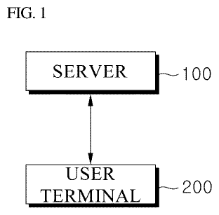

FIG. 1 is a diagram illustrating a configuration of a system including the electronic device according to an embodiment of the present disclosure.

As illustrated in FIG. 1, the system according to the embodiment of the present disclosure may include a server (100) and a user terminal (200). The electronic device according to the embodiment of the present disclosure may refer to the server (100), and may also refer to the user terminal (200) operated in conjunction with the server (100). However, for convenience of description, hereinafter, the server (100) will be referred to as the electronic device according to the embodiment of the present disclosure.

The server (100) as the electronic device according to the embodiment of the present disclosure may be operated in conjunction with the user terminal (200) as an external device through a communication function, and the server (100) and the user terminal (200) may transmit and receive various data.

According to an embodiment, the server (100) may receive food photos taken before and after a meal from the user terminal (200), and may perform an artificial intelligence model-based analysis on the photos. In this case, the server (100) may determine food intake information, such as a type of food consumed by a user and a food intake amount, through the food photos taken before and after the meal.

The server (100) may monitor health conditions of the user, based on the food intake information of the user. For example, the server (100) may determine the amount of calories consumed by the user, a lack of specific nutrients, or the like, based on the food intake information, and may support a notification operation for a warning when it is determined that a determination result falls below a reference range or exceeds the reference range.

In addition, the server (100) may support a notification operation to warn the user when it is determined that a problem situation continues for a reference period or longer through accumulation of the food intake information of the user.

In addition, the server (100) may analyze the food intake information of the user, may generate guide information based on an analysis result, and may transmit the guide information to the user.

The server (100) may transmit the analysis result of the food intake information identified through the food images before and after the meal of the user to the user terminal (200).

Accordingly, the user terminal (200) may display and provide the user with information obtained by analyzing the food intake information of the user.

Meanwhile, the electronic device according to the embodiment of the present disclosure may mean a device in which a component (for example, a display unit) for providing information to the user is included in the server (100), instead of a form separated from the user terminal as illustrated in FIG. 1.

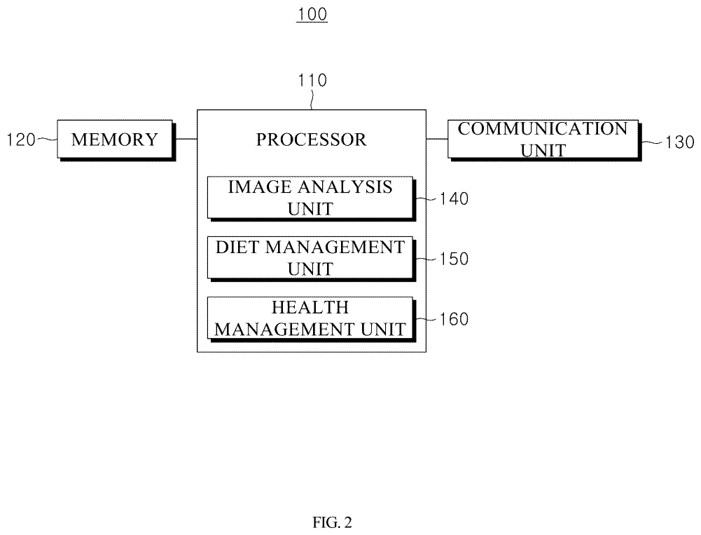

Hereinafter, a configuration of the server (100) will be described in detail with reference to FIGS. 2 to 4.

FIG. 2 is a diagram illustrating a configuration of the electronic device (server) according to an embodiment of the present disclosure.

As illustrated in FIG. 2, the server (100) according to the embodiment of the present disclosure may include a processor (110), a memory (120), and a communication unit (130).

The processor (110) may include an image analysis unit (140), a diet management unit (150), and a health management unit (160).

Prior to description of the processor (110), the memory (120) and the communication unit (130) will be described.

The memory (120) may store an AI model for identifying the food intake information from food images before and after a meal.

In addition, the memory (120) may acquire and store basic information required for determining the health conditions of the user, such as a currently held disease of the user and an age of the user.

In addition, the memory (120) may store information relating to nutritional intake which needs to be taken into account in response to disease information of the user. The communication unit (130) may receive various data transmitted from an external device (for example, the user terminal). For example, the communication unit (130) may receive personal information of the user (for example, a currently held disease, an age group, a gender, and the like), the food images before and after the meal, or the like.

In addition, the communication unit (130) may transmit various information (for example, a recommended diet, a warning message, and the like) obtained by analyzing the food intake information of the user from the server (100) to an external device (user terminal).

The processor (110) may identify the food intake information of the user from the food images before and after the meal, may analyze the identified food intake information, and may monitor the health conditions of the user.

The processor (110) may include an image analysis unit (140), a diet management unit (150), and a health management unit (160).

The image analysis unit (140) acquires the food images captured by the user before and after the meal, and may extract the food intake information of the user (for example, a type of food consumed by the user or the amount of food consumed by the user), based on a difference identified by comparison therebetween.

In this stage, the image analysis unit (140) may first determine whether the food image acquired from the user side satisfies preset requirements for identifying the food intake information.

For example, the image analysis unit (140) may determine whether the food images acquired from the user side satisfy a reference for a minimum number (for example, 2 images or more). In this case, the two images serving as the reference respectively correspond to the food image before the meal and the food image after the meal.

In addition, the image analysis unit (140) may confirm whether containers containing the food are identical in the acquired food images before the meal and after the meal. In this case, when it is determined that the containers containing the food are not identical between the food images before and after the meal, the image analysis unit (140) may determine that the images do not satisfy the requirements for identifying the food intake information.

In addition, the image analysis unit (140) may confirm whether the acquired food images before the meal and after the meal satisfy the requirement for identifying a volume of the container containing the food, and may request the user to capture an image to satisfy the requirement. For example, in order to determine the volume of the container containing the food, the image analysis unit (140) may request the user to capture the image at an angle at which a depth and a width of the container containing the food are confirmed (for example, corresponding to a visual angle at which three axes (horizontal axis, vertical axis, and height) of an object are each tilted at the same angle or a visual angle the same as a visual angle of an isometric perspective view at an angle of approximately 120 degrees). For example, the image analysis unit (140) may provide guide information on an imaging angle on an imaging screen in a stage of supporting the user to capture the images before the meal and after the meal. Accordingly, the image analysis unit (140) may help the user to capture the image at an angle at which the depth and the width of the container containing the food are confirmed.

Alternatively, along with the image before the meal which is captured at any angle, the image analysis unit (140) may request the user to additionally input the food image captured at an angle at which the width of the container is confirmed (for example, the image captured at a visual angle at which the container is viewed from above, a visual angle the same as a visual angle of a top perspective view), and the food image (for example, a side image) captured at an angle at which the depth of the container is confirmed. In addition, the image analysis unit (140) may determine whether the food image acquired through various items satisfies the requirements for identifying the food intake information. For example, the image analysis unit (140) may determine that the corresponding image does not satisfy the requirements for identifying the food intake information when a new menu which is not identified in the image before the meal is identified in the image after the meal, or when menu identity between the images before and after the meal is equal to or smaller than a reference value.

In this case, when the image analysis unit (140) determines that the acquired food image does not satisfy the above-described requirements, the image analysis unit (140) may re-request for reception of the food image serving as an analysis target (may request to change the image) or may guide items to be corrected to satisfy the above-described requirements.

When the acquired food image satisfies the requirements for identifying the food intake information, the image analysis unit (140) may input the food images acquired from the user before and after the meal into an artificial intelligence model, and may identify information on a type of the food and a mass of the food in the food image before the meal, and a type of the food and a mass of the food for which it is determined that the user takes in the food, based on an operation of the artificial intelligence model.

In this case, the artificial intelligence model may identify a volume of the food contained in the container from the food image, and may derive information on the mass of the food from the identified volume.

Specifically, the artificial intelligence model may include a first model that performs a first operation for identifying the volume of the container from the food image input by the user, and a second model that performs a second operation for identifying the volume of the food, based on a degree of the food filling the container.

In order to perform these operations, the artificial intelligence model (first model) may be trained, based on an images in a state where various types of the food are contained in the containers and information on an actual volume of the food for each image.

In this way, the first model may identify the volume of the food later, based on a difference in the degree of the food filling the container which is confirmed in the image before the meal and the image after the meal.

In this way, the artificial intelligence model may identify the volume of the container, and may estimate each of an initial volume of the food and the volume of the food consumed by the user, based on the degree of the food filling the container and the difference in the degree of the food filling the container.

The artificial intelligence model may include the second model trained to identify a type of the food from the food image before the food is consumed by the user. The second model may be trained through various food images and label information corresponding to food items in each image. Accordingly, the artificial intelligence model may perform an operation for identifying the type of the food from any food image. Specifically, the artificial intelligence model (second model) may be trained to detect and classify the type of the food, based on an object recognition algorithm (for example, YOLO, Faster R-CNN, or the like).

Information on the type of the food which is derived in this way is required to derive density for each type of the food.

The image analysis unit (140) may derive the density of each type of the food by corresponding to a composition of ingredients included in each type of the food and a ratio of each ingredient.

According to an embodiment, the image analysis unit (140) may identify information on basic composition ingredients and the ratio of each ingredient for each type of the food in accordance with a pre-stored table, and the density (for example, basic density) of the food which is derived information.

The image analysis unit (140) may calculate a basic mass value, based on the density (volume mass ratio) preset for each ingredient.

However, without being limited thereto, the image analysis unit (140) may derive a correction value obtained by correcting the basic mass value, based on a cooking method.

The image analysis unit (140) may use a method for identifying the cooking method to identify the cooking method, based on information on the type of the identified food, or may identify the cooking method through an artificial intelligence model (for example, a third model) trained to identify the cooking method, based on the food image.

First, the image analysis unit (140) may derive the cooking method for each type of the food through a pre-stored table or a data search method in which information on the cooking method for each type of the food is matched and recorded.

Alternatively, the image analysis unit (140) may identify the cooking method by inputting the food image into the artificial intelligence model (third model) trained to determine the cooking method from the food image.

Specifically, the AI model (for example, the third model) may be trained to learn color, texture, and shape information in the food image and to recognize a pattern for each cooking method. For example, the AI model may learn an item in which fried food has a color corresponding to a golden or brown color and a crispy surface texture is confirmed, and an item in which it is confirmed that steamed food has a soft and moist texture.

Specifically, as the fried food, the artificial intelligence model (third model) may identify the food in which it is confirmed that the food has a color combination of the golden or brown color and a crispy exterior texture.

In addition, the artificial intelligence model (third model) may identify that the cooking method of the food corresponds to ‘grilling’ in a case of the food in which a dark brown or black stripe is confirmed on the food and it is confirmed that the food has the crispy exterior texture.

In addition, the artificial intelligence model (third model) may identify that the cooking method of the food corresponds to ‘stewing’ of the food in which a consistent color based on a dark brown, red, or black color is confirmed, a moist and slightly sticky surface texture is confirmed, and a shiny surface that reflects light is confirmed throughout the food.

The image analysis unit (140) may derive a mass correction value by reflecting a density change according to the cooking method (for example, frying, steaming, or grilling) derived through the third model.

According to an embodiment, the image analysis unit (140) may correct a basic mass value by using ‘(Mass correction value)=(Basic mass value)*(Coefficient according to cooking method)’.

Thereafter, the image analysis unit (140) may finally derive a correction mass value by adding the mass correction value calculated in the above-described method prior to the basic mass value.
(Correction mass value)=(Basic mass value)+(Mass correction value)

A method for deriving a coefficient according to the above-described cooking method may be as follows.

The image analysis unit (140) may set a density correction parameter according to the cooking method, based on an artificial intelligence model (for example, a fourth model) trained to derive a change in the density according to the cooking method, and in a case of the cooking method (for example, frying) in which the density is reduced, the image analysis unit (140) may derive a coefficient (for example, a coefficient according to a level of density change) according to the cooking method reflecting this density correction parameter. In this case, a degree of the change in the density may be differentially applied according to the cooking method (for example, 5% decrease for frying, 5% increase for steaming, or the like).

Furthermore, the image analysis unit (140) may identify a correction condition for the basic mass value, and may perform correction only when a correction condition is satisfied. When the correction condition is not satisfied, the image analysis unit (140) may perform a first mode (for example, a basic mode) for deriving the basic mass value without correction, without calculating the mass correction value and the correction mass value.

On the other hand, when the correction condition is satisfied, the image analysis unit (140) may perform a second mode (for example, a correction mode) for calculating the mass correction value and the correction mass value.

The image analysis unit (140) may determine that the correction condition is satisfied in at least one of the following cases. For example, when the number of types of the food identified in the food image before the meal is equal to or greater than a reference value, and when the volume information of the food (for example, volume information derived by the first model) is equal to or greater than the reference value.

In this way, the artificial intelligence model may identify the type of the food and the mass of the food consumed by the user through the food images before the meal and after the meal.

The diet management unit (150) may manage the diet of the user, based on the food intake information identified by the image analysis unit (140), based on the food images before and after the meal.

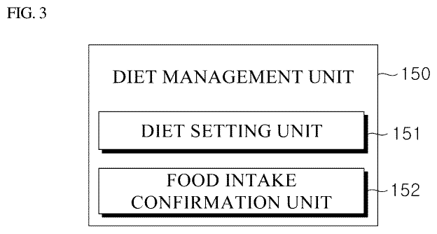

A configuration of the diet management unit (150) will be described in more detail with reference to FIG. 3.

FIG. 3 is a diagram illustrating a configuration of the diet management unit according to an embodiment of the present disclosure.

As illustrated in FIG. 3, the diet management unit (150) may include a diet setting unit (151) and a food intake confirmation unit (152).

The diet setting unit (151) may set a target diet corresponding to a need of the user. For example, the diet setting unit (151) may set the diet in response to the need of the user which relates to weight control, such as a weight gain or a weight loss.

Accordingly, the diet setting unit (151) may generate target diet information, based on target food intake information (for example, a target intake calorie, a volume of each target intake nutritional ingredient, or the like) directly input by the user.

In this case, the diet setting unit (151) may acquire the food intake information accumulated for a reference period or longer of the user before setting the target diet suitable for the user, and may determine information on a usual diet of the user through this food intake information. Thereafter, the diet setting unit (151) may generate the target diet to which an increase value or a decrease value is applied for at least one food intake item (for example, a calorie, a specific nutritional ingredient, or the like) in response to the need of the user from the usual diet of the user.

The food intake confirmation unit (152) may calculate information on the nutritional ingredients consumed by the user (type and volume of the nutritional ingredients) from the food intake information confirmed from the images of the user before and after the meal.

The food intake confirmation unit (152) may determine whether the nutritional ingredients consumed by the user are appropriate. In this case, the food intake confirmation unit (152) may determine whether the nutritional ingredients are the appropriate, based on the target diet of the user which is generated by the diet setting unit (151) or standard diet information applied to all users.

The health management unit (160) may monitor the health conditions of the user, based on the food intake information of the user. Specifically, the health management unit (160) may determine whether the nutritional intake is appropriate, based on the food intake information of the user (type of the food consumed by the user or intake amount of each food), and may guide information on an expected potential disease, based on the food intake information of the user.

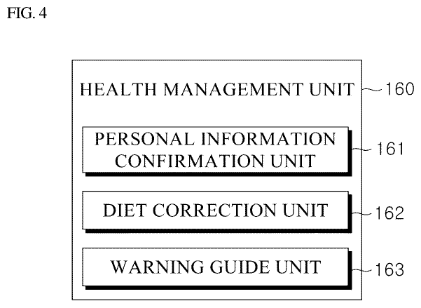

The health management unit (160) will be described in more detail with reference to FIG. 4.

FIG. 4 is a diagram illustrating a configuration of the health management unit according to an embodiment of the present disclosure.

As illustrated in FIG. 4, the health management unit (160) may include a personal information confirmation unit (161), a diet correction unit (162), and a warning guide unit (163).

The personal information confirmation unit (161) may acquire user basic information. For example, the user basic information may include at least one of physical measurement information of the user (height, weight, waist circumference, and the like), a currently held disease, a gender, and an age.

The personal information confirmation unit (161) may perform a primary prediction to predict a potential disease in which a probability of a developing disease is equal to or greater than a reference value from the user basic information.

In this case, the potential disease may be disease information derived from the currently held disease of the user or occurring as the currently held disease develops, and may mean a type of disease information different from the currently held disease.

The personal information confirmation unit (161) may perform a second prediction to calculate a ranking according to the risk of developing the potential disease and the potential disease corresponding to a preset reference ranking (for example, first place, first to third places, and the like) by combining the information on the potential disease derived from the first prediction and information on the usual diet information of the user.

Accordingly, the personal information confirmation unit (161) may acquire the user basic information directly input from the user, a first prediction value calculated according to the first prediction performed from the user basic information, and a second prediction value calculated according to the second prediction performed from the user basic information and the food intake information of the user.

The diet correction unit (162) may correct the target diet, based on information (user basic information, first prediction value, and second prediction value) acquired from the personal information confirmation unit (161). Accordingly, the diet correction unit (162) may configure the diet required for preventing the disease for which a probability of a developing disease is high, based on personal physical characteristics of the user and usual eating habits. In addition, since the diet may be corrected based on the target diet previously generated by the user in response to his or her own need, the user may be guided to the diet configured in view of both his or her needs relating to the weight and a health maintaining purpose.

The warning guide unit (163) may determine whether the type and the amount of the nutritional ingredients consumed by the user fall within an appropriate range, based on the food intake information of the user. In this case, the warning guide unit (163) may set a reference for determining the appropriateness of the nutritional information of the user (type of the nutritional ingredients and each nutritional ingredient intake amount) consumed by the user, as the target diet of the user. The target diet may include information on the type and the amount of the nutritional ingredients recommended for the user to take in the nutritional ingredients per meal or per day.

As a result of determining whether the nutritional information consumed by the user is appropriate, the warning guide unit (163) may generate a warning message and may provide a notification to the user when it is determined that the nutritional information consumed by the user is inappropriate.

For example, the warning guide unit (163) may compare the intake amount of each type of the nutritional ingredients recommended to the user according to the target diet of the user with the intake amount of each type of the nutritional ingredients determined to be consumed by the user, and when a difference exceeds the reference value, the warning guide unit (163) may determine that the nutritional intake of the user is inappropriate, and may issue a warning of the inappropriate nutritional intake.

In addition, the warning guide unit (163) may evaluate the appropriateness of the nutritional intake of the user, based on not only the target diet but also usually recommended standard diet information.

In addition, the warning guide unit (163) may receive an input of the food image twice from the user before the meal and after the meal. After receiving the input of the food image before the meal, the warning guide unit (163) may identify a restricted food intake item, and may generate a warning message to guide the user.

For example, the warning guide unit (163) may determine that the food intake amount of the user already exceeds the daily intake amount of specific nutritional ingredients (for example, sugar) at lunch, based on recommended nutritional information set based on the target diet or the like. In addition, when a menu in which the nutritional ingredients are expected to exceed the reference value is confirmed in the food image before dinner, the warning guide unit (163) may generate a warning message (for example, do not drink fruit juice) to limit the intake of the corresponding menu, and may provide the warning message to the user.

In short, the electronic device according to an embodiment of the present disclosure may include the memory that stores the AI model for identifying the food intake information from the food images before and after the meal, and the processor that inputs the food images before and after the meal into the AI model to identify the food intake information of the user, and that monitors the health conditions of the user, based on the food intake information.

The processor may determine whether the food images before and after the meal satisfy the requirements for identifying the food intake information, based on whether the containers and the food menus which are included in each of the food images before and after the meal are identical, and may request the user to change the food images before and after the meal when the requirements are not satisfied.

In addition, in a process of identifying the food intake information of the user from the food images before and after the meal, the processor may identify the mass of the food consumed by the user, based on volume information on the food reduced according to a difference between the food image before the meal and the food image after the meal, and may determine the mass of the food consumed by the user by applying the density according to the type of the food to the volume information on the reduced food.

In addition, the processor may identify the mass of the food consumed by the user, based on the volume information on the food reduced by the difference between the food image before the meal and the food image after the meal, and may determine the mass of the food consumed by the user by reflecting a change in the density according to the cooking method of the food.

In addition, the processor may set the target diet in response to a weight control need of the user, and may generate the target diet in which a numerical value of at least one intake item is increased or decreased in response to the need from the usual diet of the user.

In addition, the processor may determine information on the usual diet of the user, based on the food intake information of the user which is accumulated for a reference period or longer.

In addition, the processor may calculate the type and the volume of the nutritional ingredients consumed by the user, based on the food intake information, and may determine whether the type and the volume of the nutritional ingredients consumed by the user are appropriate.

In addition, the processor may acquire the user basic information including at least one of the body measurement information of the user, the currently held disease, the gender, and the age, may predict the potential disease in which a probability of a developing disease is equal to or greater than a reference value from the user basic information, and may generate the target diet to prevent the potential disease.

In addition, a system according to an embodiment of the present disclosure may include the user terminal that generates the food images before and after the meal through image capturing, and the electronic device that receives the generated food images from the user terminal to analyze the generated food images. The electronic device may include the memory that stores the AI model for identifying the food intake information from the food images before and after the meal, and the processor that inputs the food images before and after the meal into the AI model to identify the food intake information of the user, and that monitors the health conditions of the user, based on the food intake information.

A control method for the electronic device according to an embodiment of the present disclosure may include a step of acquiring the food images before and after the meal, a step of identifying the food intake information of the user by inputting the food images before and after the meal into the AI model, and a step of monitoring the health conditions of the user, based on the food intake information.

In addition, there is provided a computer readable medium storing the program that causes the processor of the electronic device to execute the control method.

The electronic device (100) according to an embodiment of the present disclosure may include the processor (110), the memory (120), the communication unit (130), and the like.

The memory may store various programs and data required for operating the electronic device. The memory may be implemented as a non-volatile memory, a volatile memory, a flash-memory, a hard disk drive (HDD), or solid-state drive (SSD).

The communication unit may communicate with an external device. In particular, the communication unit may include various communication chips such as a WiFi chip, a Bluetooth chip, a wireless communication chip, an NFC chip, a low-power Bluetooth chip (BLE chip), and the like. In this case, the WiFi chip, the Bluetooth chip, and the NFC chip respectively perform communication in a LAN mode, a WiFi mode, a Bluetooth mode, and an NFC mode. When the WiFi chip or the Bluetooth chip is used, various connection information such as an SSID and a session key are first transmitted and received, communication is connected by using this information, and thereafter, various information may be transmitted and received. The wireless communication chip refers to a chip that performs communication according to various communication standards such as IEEE, Zigbee, 3rd Generation (3G), 3rd Generation Partnership Project (3GPP), and Long Term Evolution (LTE).

The processor may control an overall operation of a user device by using various programs stored in the memory. The processor may include a RAM, a ROM, a graphics processing unit, a main CPU, first to n-th interfaces, and a bus. In this case, the RAM, the ROM, the graphics processing unit, the main CPU, the first to n-th interfaces, and the like may be connected to each other via the bus.

The RAM stores an O/S and application programs. Specifically, when the electronic device boots up, the O/S may be stored in the RAM, and various application data selected by the user may be stored in RAM.

The ROM stores a set of commands for system booting. When a turn-on command is input to supply power, the main CPU copies the O/S stored in the memory (200) to the RAM in accordance with the command stored in the ROM, and executes the O/S to boot the system. When the booting is complete, the main CPU copies various application programs stored in the memory to the RAM, and executes the application programs copied to the RAM to perform various operations.

The main CPU accesses the memory, and performs operations including the booting and the execution by using the OS stored in the memory. In addition, the main CPU performs various operations by using various programs, contents, and data which are stored in the memory.

The first to n-th interfaces are connected to the various components described above. One of the first to n-th interfaces may be a network interface connected to an external device via a network.

Meanwhile, furthermore, the processor may control the artificial intelligence model. In this case, as a matter of course, the control unit may include a processor dedicated to graphics (for example, a GPU) for controlling the artificial intelligence model.

The processor may include one or more cores (not illustrated) and a graphics processing unit (not illustrated) and/or a connection path (for example, a bus) for transmitting and receiving signals with other components.

The processor according to an embodiment performs the method described with reference to the present disclosure by executing one or more instructions stored in the memory. Meanwhile, the processor may further include a Random Access Memory (RAM, not illustrated) and a Read-Only Memory (ROM, not illustrated) which temporarily and/or permanently store signals (or data) processed inside the processor. In addition, the processor (130) may be implemented in a form of a system on chip (SoC) including at least one of the graphics processing unit, the RAM, and the ROM.

The memory may store programs (one or more instructions) for processing and controlling the processor. The programs stored in the storage may be divided into a plurality of modules depending on functions.

The steps of the method or the algorithm described with reference to the embodiments of the present disclosure may be implemented directly in hardware, in a software module executed by the hardware, or in a combination thereof. The software module may reside in the Random Access Memory (RAM), the Read Only Memory (ROM), an Erasable Programmable ROM (EPROM), an Electrically Erasable Programmable ROM (EEPROM), a Flash Memory, a hard disk, a removable disk, a CD-ROM, or any other form of computer readable recording medium well known in the art to which the present disclosure belongs.

The components of the present disclosure may be implemented as the program (or application) to be executed in combination with a hardware computer, and may be stored in a medium. The components of the present disclosure may be implemented as software programming or software elements. Similarly, the embodiments may be implemented in a programming or scripting language, such as C, C++, Java, and an assembler, including various algorithms implemented as a combination of data structures, processes, routines or other programming configurations. Functional aspects may be implemented as algorithms executed by one or more processors.

Although the present disclosure has been described in detail with reference to the examples described above, those skilled in the art may make modifications, changes, and variations to the examples without departing from the scope of the present disclosure. In short, in order to achieve the intended effect of the present disclosure, it is not necessary to separately include all of the functional blocks illustrated in the drawings or to exactly follow all of the orders illustrated in the drawings as the illustrated order. Even in other cases, it should be noted that the modifications, the changes, and the variations may fall within the technical scope of the present disclosure set forth in the appended claims.

### DETAILED DESCRIPTION OF MAIN ELEMENTS

100: electronic device (server)
110: processor
120: memory
130: communication unit
140: image analysis unit
150: diet management unit
160: health management unit
200: user terminal

What is claimed is:

1. An electronic device comprising:
a memory that stores an AI model for identifying food intake information of a user from food images before and after a meal; and
a processor that inputs the food images before and after the meal into the AI model to identify the food intake information of the user, and that monitors health conditions of the user, based on the food intake information.

2. The electronic device of claim 1, wherein the processor determines whether the food images before and after the meal satisfy requirements for identifying the food intake information, based on whether containers and food menus which are included in each of the food images before and after the meal are identical, and requests the user to change the food images before and after the meal, when the requirements are not satisfied.

3. The electronic device of claim 1, wherein in a process of identifying the food intake information of the user from the food images before and after the meal, the processor identifies a mass of food consumed by the user, based on volume information of the reduced food according to a difference between the food image before the meal and the food image after the meal, and determines the mass of the food consumed by the user by applying density depending on a type of the food to the volume information of the reduced food.

4. The electronic device of claim 3, the processor identifies the mass of the food consumed by the user, based on the volume information of the reduced food according to the difference between the food image before the meal and the food image after the meal, and determines the mass of the food consumed by the user by reflecting a change in the density according to a cooking method of the food.

5. The electronic device of claim 1, wherein the processor sets a target diet in response to a weight control need of the user, and generates the target diet in which a numerical value of at least one intake item is increased or decreased from a usual diet of the user, in response to the need.

6. The electronic device of claim 5, wherein the processor determines information on the usual diet of the user, based on the food intake information of the user which is accumulated over a reference period.

7. The electronic device of claim 1, wherein the processor calculates a type and a volume of nutritional ingredients consumed by the user, based on the food intake information, and determines whether the type and the volume of the nutritional ingredients consumed by the user are appropriate.

8. The electronic device of claim 1, wherein the processor acquires user basic information including at least one of body measurement information, a currently held disease, a gender, and an age of the user, predicts a potential disease with a risk of developing disease is equal to or greater than a reference value from the user basic information, and generates the target diet to prevent the potential disease.

9. A system comprising:
a user terminal that generates food images before and after a meal through image capturing; and
an electronic device that receives the generated food images from the user terminal to analyze the generated food images,
wherein the electronic device includes a memory that stores an AI model for identifying food intake information of a user from the food images before and after the meal, and a processor that inputs the food images before and after the meal into the AI model to identify the food intake information of the user, and that monitors health conditions of the user, based on the food intake information.

10. A control method for an electronic device, comprising:
acquiring food images before and after a meal;
identifying food intake information of a user by inputting the food images before and after the meal into an AI model; and
monitoring health conditions of the user, based on the food intake information.

11. A computer readable medium storing a program that causes a processor of an electronic device to execute the control method according to claim 10.
